(19) United States
(12) Patent Application Publication
(10) Pub. No.: US 2026/0188438 A1
(43) Pub. Date: Jul. 2, 2026

(54) METHOD AND DEVICE WITH SEQUENTIAL DATA PROCESSING

(71) Applicant: Samsung Electronics Co., Ltd., Suwon-si (KR)

(72) Inventors: Seongeon PARK, Suwon-si (KR); Hyohyeong KANG, Suwon-si (KR); Hogyeong KIM, Suwon-si (KR); Jinwoo PARK, Suwon-si (KR); Myeong In KANG, Suwon-si (KR); Young-Seok KIM, Suwon-si (KR); Jooyeon AHN, Suwon-si (KR); Seonwoo LEE, Suwon-si (KR)

(73) Assignee: Samsung Electronics Co., Ltd., Suwon-si (KR)

(21) Appl. No.: 19/261,868

(22) Filed: Jul. 7, 2025

(30) Foreign Application Priority Data
Dec. 30, 2024 (KR) 10-2024-0201214

(51) Int. Cl. G16C 20/70; G16C 20/30

(52) U.S. Cl. CPC G16C 20/70; G16C 20/30

(57) ABSTRACT

A processor-implemented method includes extracting features from action data included in a synthesis process, the action data being in a time order, generating a vector sequence corresponding to the action data based on embedding data of the features extracted from the action data, and generating data regarding the synthesis process generated from encoded data of the vector sequence, by applying the vector sequence to a sequential data processing model.

### CROSS-REFERENCE TO RELATED APPLICATIONS

This application claims the benefit under 35 USC § 119(a) of Korean Patent Application No. 10-2024-0201214, filed on Dec. 30, 2024 in the Korean Intellectual Property Office, the entire disclosure of which is incorporated herein by reference for all purposes.

### BACKGROUND

### 1. Field

The following description relates to a method and device with sequential data processing.

### 2. Description of Related Art

Technology to simulate a recipe of processing or synthesizing material aims to simulate various physical and chemical parameters in a digital environment. Through this, optimal processing conditions and synthesis results may be predicted without actual experiments, and various data may be obtained for efficient execution of experiments. Machine learning and big data analysis may be integrated to further improve the prediction accuracy of simulations and may be used in various industrial fields such as material development, semiconductors, and pharmaceuticals.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one or more general aspects, a processor-implemented method includes extracting features from action data included in a synthesis process, the action data being in a time order, generating a vector sequence corresponding to the action data based on embedding data of the features extracted from the action data, and generating data regarding the synthesis process generated from encoded data of the vector sequence, by applying the vector sequence to a sequential data processing model.

The generating of the data regarding the synthesis process may include generating material property data that is predicted as a result of the synthesis process, by applying the vector sequence to the sequential data processing model.

The generating of the vector sequence may include generating the embedding data of the features extracted from the action data, based on preprocessing of the features extracted from the action data.

The preprocessing of the features may include any one or any combination or any two or more of determining a value of an unextracted feature, normalizing numerical features, and encoding categorical features.

The sequential data processing model may include a transformer-based model trained to predict a material property of an output of an input process from a vector sequence corresponding to the input process.

The extracting of the features from the action data may include extracting the features from the action data for each time step included in the synthesis process, the synthesis process being time-series data.

The extracting of the features may include extracting a predetermined type of feature from each piece of the action data.

The generating of the vector sequence may include generating, as the embedding data, embedding data of each feature extracted from the action data, generating a vector corresponding to each piece of the action data by aggregating the embedding data of each feature, and generating the vector sequence comprising the vector corresponding to each piece of the action data.

The generating of the vector corresponding to each piece of the action data may include generating a first aggregate feature by aggregating embedding data corresponding to one or more features extracted from each piece of the action data, generating a second aggregate feature by aggregating embedding data corresponding to one or more features extracted from each piece of the action data, and aggregating the first aggregate feature and the second aggregate feature.

The sequential data processing model may include a first head for material property prediction, and a second head for uncertainty prediction, and the generating of the data regarding the synthesis process may include generating an output of the first head and an output of the second head corresponding to the encoded data of the vector sequence, and determining, from a synthesis process database, another synthesis process to be input to the sequential data processing model, based on an acquisition function predefined for the output of the first head and the output of the second head.

The method may include training an autoencoder based on sequential data processing to output a restored vector sequence of the vector sequence based on the encoded data of the vector sequence.

In one or more general aspects, a processor-implemented method includes extracting features from action data in a time order included in a synthesis process, generating a vector sequence corresponding to the action data based on embedding data of the features extracted from the action data, and training an autoencoder based on sequential data processing to output a restored vector sequence of the vector sequence based on encoded data of the vector sequence.

The training of the autoencoder may include training an encoder of the autoencoder to generate the encoded data of the vector sequence, and for the outputting of the restored vector sequence, training a decoder of the autoencoder to restore the vector sequence from the encoded data of the vector sequence generated by the encoder.

The training of the autoencoder may include applying, to a decoder of the autoencoder, a latent vector sampled from a latent space of the vector sequence output from an encoder of the autoencoder, based on noise obtained according to a predetermined probability distribution, and for the outputting of the restored vector sequence, training the decoder of the autoencoder to restore the vector sequence from the latent vector.

The decoder of the trained autoencoder may be configured to generate a vector sequence corresponding to an arbitrary synthesis process from arbitrary noise obtained according to a probability distribution.

In one or more general aspects, an electronic device includes one or more processors comprising processing circuitry, and memory comprising one or more storage media storing instructions that, when executed individually or collectively by the one or more processors, cause the electronic device to extract features from action data included in a synthesis process, the action data being in a time order, generate a vector sequence corresponding to the action data based on embedding data of the features extracted from the action data, and generate data regarding the synthesis process generated from encoded data of the vector sequence, by applying the vector sequence to a sequential data processing model.

For the generating of the data regarding the synthesis process, the execution of the instructions may cause the electronic device to generate material property data that is predicted as a result of the synthesis process, by applying the vector sequence to the sequential data processing model.

For the generating of the vector sequence, the execution of the instructions may cause the electronic device to generate the embedding data of the features extracted from the action data, based on preprocessing of the features extracted from the action data.

For the extracting of the features from the action data, the execution of the instructions may cause the electronic device to extract the features from the action data for each time step included in the synthesis process, the synthesis process being time-series data.

The sequential data processing model may include a first head for material property prediction, and a second head for uncertainty prediction, and for the generating of the data regarding the synthesis process, the execution of the instructions may cause the electronic device to generate an output of the first head and an output of the second head corresponding to the encoded data of the vector sequence, and determine, from a synthesis process database, another synthesis process to be input to the sequential data processing model, based on an acquisition function predefined for the output of the first head and the output of the second head.

Other features and aspects will be apparent from the following detailed description, the drawings, and the claims.

### BRIEF DESCRIPTION OF THE DRAWINGS

FIG. 1 illustrates a flowchart of an example of an operation of a sequential data processing method corresponding to a synthesis process, according to one or more embodiments.

FIG. 2 illustrates an example of a sequential data processing method through an example of data, according to one or more embodiments.

FIGS. 3A and 3B each illustrate an example of feature data output from action data, according to one or more embodiments.

FIGS. 4A and 4B each illustrate an example of an operation of generating an embedding vector of a feature extracted from action data, according to one or more embodiments.

FIG. 5 illustrates an example of a transformer-based sequential data processing model, according to one or more embodiments.

FIG. 6 illustrates an example of an operation of extracting features from action data for each time step, according to one or more embodiments.

FIG. 7 illustrates an example of a method of determining a synthesis process to be input to a sequential data processing model from a synthesis process database, according to one or more embodiments.

FIGS. 8A and 8B each illustrate an example of an autoencoder based on sequential data processing, according to one or more embodiments.

FIG. 9 illustrates an example of a configuration of an electronic device, according to one or more embodiments.

Throughout the drawings and the detailed description, unless otherwise described or provided, the same drawing reference numerals may be understood to refer to the same or like elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences within and/or of operations described herein are merely examples, and are not limited to those set forth herein, but may be changed as will be apparent after an understanding of the disclosure of this application, except for sequences within and/or of operations necessarily occurring in a certain order. As another example, the sequences of and/or within operations may be performed in parallel, except for at least a portion of sequences of and/or within operations necessarily occurring in an order, e.g., a certain order. Also, descriptions of features that are known after an understanding of the disclosure of this application may be omitted for increased clarity and conciseness.

With regard to the description of the drawings, similar reference numerals may be used to refer to similar or related components. It is to be understood that a singular form of a noun corresponding to an item may include one or more of the things, unless the relevant context clearly indicates otherwise.

As used herein, the term “and/or” includes any one and any combination of any two or more of the associated listed items. The phrases “at least one of A, B, and C”, “at least one of A, B, or C”, and the like are intended to have disjunctive meanings, and these phrases “at least one of A, B, and C”, “at least one of A, B, or C” (e.g., each phrase may include any one of the respective items alone, all of the items listed together, and all possible combinations thereof), and the like also include examples where there may be one or more of each of A, B, and/or C (e.g., any combination of one or more of each of A, B, and C), unless the corresponding description and embodiment necessitates such listings (e.g., “at least one of A, B, and C”) to be interpreted to have a conjunctive meaning.

Although terms such as “first,” “second,” and “third”, or A, B, (a), (b), and the like may be used herein to describe various members, components, regions, layers, or sections, these members, components, regions, layers, or sections are not to be limited by these terms. Each of these terminologies is not used to define an essence, order, or sequence of corresponding members, components, regions, layers, or sections, for example, but used merely to distinguish the corresponding members, components, regions, layers, or sections from other members, components, regions, layers, or sections. Thus, a first member, component, region, layer, or section referred to in the examples described herein may also be referred to as a second member, component, region, layer, or section without departing from the teachings of the examples.

Throughout the specification, when a component or element is described as being “on”, “connected to,” “coupled to,” or “joined to” another component, element, or layer it may be directly (e.g., in contact with the other component, element, or layer) “on”, “connected to,” “coupled to,” or “joined to” the other component, element, or layer or there may reasonably be one or more other components, elements, layers intervening therebetween. When a component, element, or layer is described as being “directly on”, “directly connected to,” “directly coupled to,” or “directly joined” to another component, element, or layer there can be no other components, elements, or layers intervening therebetween. Likewise, expressions, for example, “between” and “immediately between” and “adjacent to” and “immediately adjacent to” may also be construed as described in the foregoing.

The terminology used herein is for describing various examples only and is not to be used to limit the disclosure. The articles “a,” “an,” and “the” are intended to include the plural forms as well, unless the context clearly indicates otherwise. As non-limiting examples, terms “comprise” or “comprises,” “include” or “includes,” and “have” or “has” specify the presence of stated features, numbers, operations, members, elements, and/or combinations thereof, but do not preclude the presence or addition of one or more other features, numbers, operations, members, elements, and/or combinations thereof, or the alternate presence of an alternative stated features, numbers, operations, members, elements, and/or combinations thereof. Additionally, while one embodiment may set forth such terms “comprise” or “comprises,” “include” or “includes,” and “have” or “has” specify the presence of stated features, numbers, operations, members, elements, and/or combinations thereof, other embodiments may exist where one or more of the stated features, numbers, operations, members, elements, and/or combinations thereof are not present.

Unless otherwise defined, all terms, including technical and scientific terms, used herein have the same meaning as commonly understood by one of ordinary skill in the art to which this disclosure pertains and specifically in the context on an understanding of the disclosure of the present application. Terms, such as those defined in commonly used dictionaries, are to be interpreted as having a meaning that is consistent with their meaning in the context of the relevant art and specifically in the context of the disclosure of the present application, and are not to be interpreted in an idealized or overly formal sense unless expressly so defined herein.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application. The use of the term “may” herein with respect to an example or embodiment (e.g., as to what an example or embodiment may include or implement) means that at least one example or embodiment exists where such a feature is included or implemented, while all examples are not limited thereto. The use of the terms “example”, “embodiment”, and “example embodiment” herein have a same meaning (e.g., the phrasing ‘in an or one example’ has a same meaning as ‘in an or one embodiment“ and ‘in an or one example embodiment’), and ”one or more examples“ has a same meaning as ”one or more embodiments“ and ”one or more example embodiments“. Still further, each of multiple or all separately described an/one ”example“, ”embodiment“, ”example embodiment“, as well as ”examples“, ”embodiments“, ”example embodiments“, herein may be included, in combination, in a same embodiment in any combination.

Hereinafter, the embodiments are described in detail with reference to the accompanying drawings. When describing the embodiments with reference to the accompanying drawings, like reference numerals refer to like components and a repeated description related thereto is omitted.

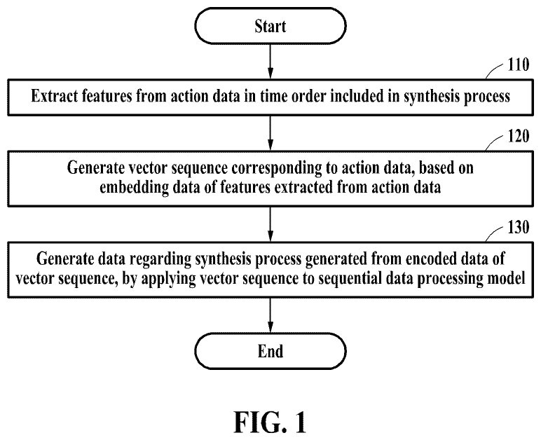

FIG. 1 illustrates a flowchart of an example of an operation of a sequential data processing method corresponding to a synthesis process, according to one or more embodiments. Operations 110 to 130 of FIG. 1 may be performed in the sequence and manner as illustrated in FIG. 1. However, one or more of the operations may be performed in a different order, one or more of the operations may be omitted, two or more of the operations may be performed in parallel or simultaneously, and/or other operations may be additionally performed without departing from the spirit and scope of the described embodiments.

A method and device of one or more embodiments may improve the efficiency of material synthesis and/or processes, and may be used to accurately predict the properties of materials. The sequential data processing method corresponding to a synthesis process may be performed in an electronic device including at least one processor. An example of a specific hardware configuration of the electronic device that performs the sequential data processing method corresponding to a synthesis process is described in detail below. Hereinafter, the sequential data processing method corresponding to a synthesis process may be briefly referred to as the sequential data processing method. While the process is described mainly as a “synthesis process” in examples herein, examples are not limited thereto, and the process may be any one of processes other than a synthesis process, according to one or more other non-limiting examples.

Referring to FIG. 1, the sequential data processing method corresponding to a synthesis process may include operation 110 of extracting features from action data, which is in a time order, included in the synthesis process.

The synthesis process may be data that indicates a process, a procedure, and/or a recipe for synthesizing a substance. The synthesis process may include, for example, any one or any combination of text data that indicates the process of synthesizing a substance and graph data that indicates changes in conditions or elements for synthesizing a substance over time.

The synthesis process may include a plurality of pieces of action data. Action data is data that instructs actions to adjust at least one condition and may include, for example, data that instructs actions such as changing temperature of an experimental environment, changing a gas composition of the experimental environment, adding a substance, and/or purifying.

A piece of action data may correspond to each step of the synthesis process. The time order may be determined between the pieces of action data. For example, the action data may include information indicating action data corresponding to a previous sequence and/or information indicating action data corresponding to a subsequent sequence.

In an embodiment, the action data may include time information. For example, the action data may include the time information indicating start and end times. For example, the action data may include information indicating duration.

One or more features may be extracted from each piece of action data. Features extracted from action data may include the attributes of information extracted from the action data and/or data that quantifies information included in the action data. For example, the features may include any one or any combination of a feature regarding the type of action included in the action data, a feature regarding a condition indicated by the action data, a feature regarding a direction of change of the condition indicated by the action data, a feature regarding a quantity of change of the condition indicated by the action data, and a feature regarding time information indicated by the action data.

In an embodiment, operation 110 of extracting features from action data may include extracting a predetermined type of feature from each piece of the action data. For example, the predetermined type of feature may include a categorical feature including at least one of an action type or a substance type. The categorical feature may be a feature that represents data as a finite number of categories or groups. A value of the categorical feature may be determined to be at least one of a plurality of labels included in a category. For example, the predetermined type of feature may include a numerical feature including at least one of a quantity of substance, temperature, duration, and/or timestamp. The numerical feature may correspond to a feature that represents data as numerical values. The value of the numerical feature may be determined to be numerical data.

As an example, a substance type feature may be divided into one or more sub-features. The substance type feature may be divided into sub-features including at least one of a cation-type feature, an anion-type feature, a solvent-type feature, and a concentration feature according to the chemical properties of a substance.

Some types of features may not be extracted from any piece of action data. A value of a feature that is not extracted from the piece of action data may be determined to be a value (e.g., null) indicating that the feature is not extracted. Specific examples of the features extracted from each piece of action data are described in detail below.

Operation 110 of extracting features from action data may include extracting features from action data for each time step included in the synthesis process, which is time-series data. The synthetic process, which is time-series data, may include data indicating changes in conditions over time and/or data indicating recorded conditions at every time step of a determined time interval (e.g., 1 second, 1 minute, 10 minutes, etc.). The action data may include data indicating conditions recorded at a time step included in the synthesis process. An example the extracting of features from action data for each time step included in the synthesis process, which is time-series data, is described in detail below.

A sequential data processing method may include operation 120 of generating, based on embedding data of a feature extracted from the action data, a vector sequence corresponding to the action data.

Feature(s) extracted from a piece of action data may be embedded to be generated as a vector of a certain size. For example, a vector, which is embedding data of a feature corresponding to each piece of action data, may be generated. When “n” pieces of action data correspond to the synthesis process, “n” vectors may be generated. A vector generated by embedding features extracted from determined action data may be referred to as a vector corresponding to that action data or a vector of that action data.

A vector sequence may include vectors corresponding to each piece of the action data. The vector sequence may be a sequence in which vectors corresponding to pieces of action data are connected in a time order between the pieces of action data. For example, when first action data, second action data, and third action data are included in the synthesis process in the above order, a first vector corresponding to the first action data, a second vector corresponding to the second action data, and a third vector corresponding to the third action data may be included in the vector sequence in the same order as the action data, that is, in the order of the first vector, the second vector, and the third vector.

Operation 120 of generating a vector sequence may include generating the embedding data of features extracted from action data, based on preprocessing of the features extracted from the action data.

For example, the preprocessing of features may include determining the value of an unextracted feature. As described above, the predetermined type of features in the action data may include an unextracted type of feature. In the preprocessing of features, the value of an unextracted feature may be determined to be a preset value (e.g., 0).

For example, the preprocessing of features may include normalizing numerical features. The scales of different types of numerical features may be transformed into a certain range of scales through normalization. For example, the numerical features may be normalized to have real numbers greater than or equal to 0 and less than or equal to 1.

For example, the preprocessing of features may include encoding categorical features. Each category of the categorical features may be mapped to a numerical value. The value of the categorical feature may be changed to the mapped numerical value. For example, when the value of a categorical feature is determined as any one of A, B, and C, A may be mapped to 0, B to 1, and C to 2. When the value of the categorical feature is A, the value of the categorical feature may be changed to 0, which is mapped to A, through preprocessing.

Operation 120 of generating a vector sequence may include generating embedding data of each feature extracted from action data, generating a vector corresponding to each piece of the action data by aggregating the embedding data of each feature, and generating the vector sequence including the vector corresponding to each piece of the action data.

The generating of a vector corresponding to each piece of the action data may include generating a first aggregate feature by aggregating embedding data corresponding to at least some of features extracted from each piece of the action data, generating a second aggregate feature by aggregating embedding data corresponding to at least some of features extracted from each piece of the action data, and aggregating the first aggregate feature and the second aggregate feature. Embedding data corresponding to at least some of the features extracted from each piece of the action data may be aggregated a plurality of times, and a plurality of aggregate features may be generated.

An example of a process of generating a vector by aggregating embedding data of each feature is described in detail below.

A sequential data processing method may include operation 130 of generating data regarding the synthesis process generated from encoded data of a vector sequence by applying the vector sequence to a sequential data processing model. The data regarding the synthesis process may include data that may be derived from the synthesis process, such as data regarding an output of the synthesis process, data for determining a next synthesis process to be input, and data that may be generated regarding the synthesis process.

Operation 130 of generating data regarding the synthesis process may include generating material property data that is predicted as a result of the synthesis process by applying the vector sequence to the sequential data processing model.

The sequential data processing model may include a model that receives sequential data including ordered data as an input and outputs a specific result. For example, the sequential data processing model may include one or more learning-based models that may process sequential data, such as a one-dimensional convolutional neural network (1d-cnn), mamba, a long short-term memory (LSTM), and/or a gated recurrent unit (GRU).

The sequential data processing model may include a model trained to predict a material property of an output of the synthesis process. For example, the sequential data processing model may include a transformer-based model trained to predict a material property of an output of an input process from a vector sequence corresponding to the input process. An example of a structure of the sequential data processing model is described in detail below.

The material property may be information indicating a quality or trait that a substance has and may include any one or any combination of quantum efficiency, synthetic yield, wavelength, quantum dot size, emission wavelength, and full width at half maximum (FWHM).

By applying the vector sequence to the sequential data processing model to generate material property data predicted as a result of the synthesis process, the method and device of one or more embodiments may generate the material property data of a substance predicted to be generated from the synthesis process without performing an actual experiment on the synthesis process.

The sequential data processing model may include a first head for material property prediction and a second head for uncertainty prediction. Operation 130 of generating data regarding the synthesis process may include generating an output of a first head and an output of a second head that correspond to encoded data of the vector sequence and determining, from a synthesis process database, a synthesis process to be input to the sequential data processing model, based on an acquisition function predefined for the output of the first head and the output of the second head.

An example of a method of determining, from the synthetic process database, a synthetic process to be input to the sequential data processing model based on an acquisition function is described in detail below.

The sequential data processing method may include training an autoencoder based on sequential data processing to output a vector sequence from the vector sequence based on the encoded data of the vector sequence. For example, the sequential data processing method may include, instead of, in addition to, or included in operation 130 of FIG. 1, an operation of training an autoencoder based on sequential data processing to output a vector sequence from the vector sequence based on the encoded data of the vector sequence.

The operation of training an autoencoder may include training an encoder of the autoencoder to generate encoded data of the vector sequence and training a decoder of the autoencoder to restore the vector sequence from the encoded data of the vector sequence generated by the encoder.

The training of the autoencoder may include applying, to a decoder of the autoencoder, a latent vector sampled from a latent space of the vector sequence output from an encoder of the autoencoder, based on noise generated according to a predetermined probability distribution, and training the decoder of the autoencoder to restore the vector sequence from the latent vector. For example, the decoder of the trained autoencoder may generate a vector sequence corresponding to an arbitrary synthesis process from arbitrary noise generated according to a probability distribution.

An example of the autoencoder based on sequential data processing is described in detail below.

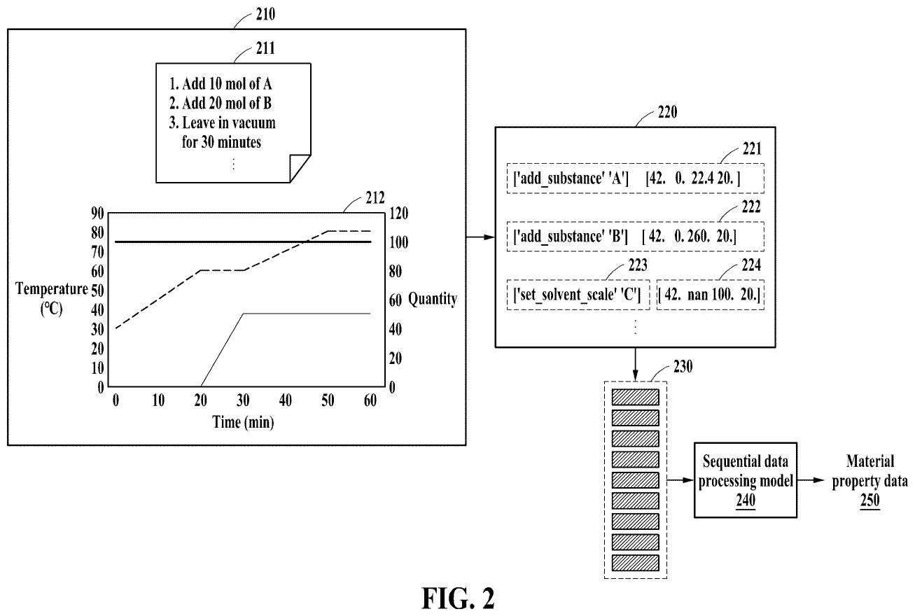

FIG. 2 illustrates an example of a sequential data processing method through an example of data, according to one or more embodiments.

Referring to FIG. 2, synthetic process data 210 may be input data of an electronic device that performs the sequential data processing method. For example, the synthesis process data 210 may include text data 211 and/or graph data 212. The text data 211 and the graph data 212 are only examples of the synthesis process data 210, and the synthesis process data 210 is not limited thereto.

The text data 211 may include data that expresses each step of a synthesis process as text. The text data 211 may include data expressed in natural language or data with text being input to a predetermined format (e.g., a table). Some texts separated by steps in the text data 211 may correspond to action data.

The graph data 212 may include data indicating changes in conditions for substance synthesis over time. For example, the graph data 212 may include graph data indicating changes in temperature over time. For example, the graph data 212 may include data indicating changes in a quantity of a substance over time. Action data may correspond to data indicating a state of a time step in the graph data 212.

Feature set data 220 may be extracted from action data in a time order included in the synthesis process. A feature set may correspond to one piece of action data. As an example, a first feature set 221 may include features extracted from first action data, and a second feature set 222 may include features extracted from second action data.

For example, the feature set data 220 may include a vector 223 listing categorical features and a vector 224 listing numerical features, in which the categorical and numerical features are extracted from each piece of action data. A feature set may include categorical features including action types and substance types. A feature set may include numerical features including time, duration, quantity of substance, and temperature.

Based on the embedding data of the features extracted from the action data, a vector sequence 230 corresponding to the action data may be generated. The vector sequence 230 may include a vector in which a feature set extracted from each piece of the action data is embedded. For example, the vector sequence 230 may include a vector in which the feature set extracted from each piece of the action data is embedded with a predetermined size.

The order between the vectors included in the vector sequence 230 may correspond to the order between the pieces of the action data. For example, an embedding vector of features extracted from action data corresponding to a first order in the synthesis process may correspond to a first order in the vector sequence 230.

The vector sequence 230 may be applied to a sequential data processing model 240 such that material property data 250 predicted as a result of the synthesis process may be generated.

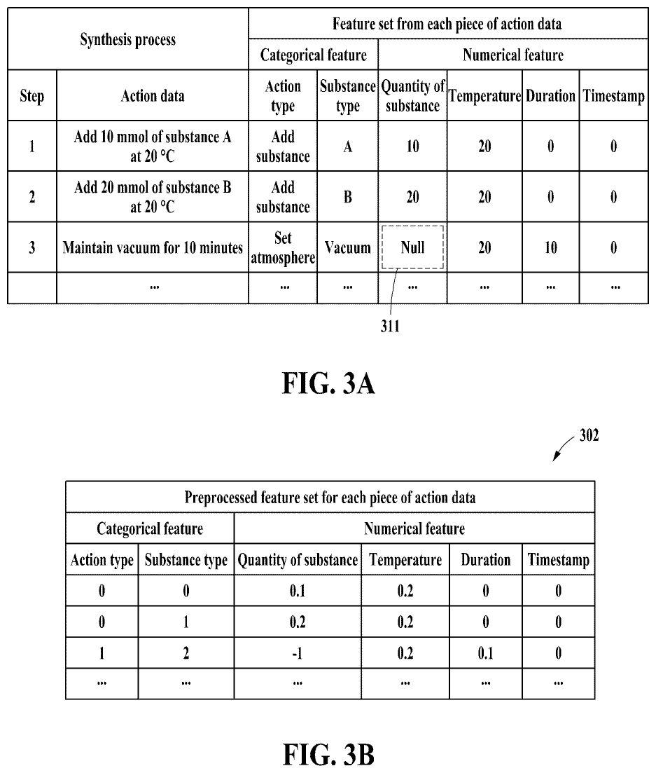

FIGS. 3A and 3B each illustrate an example of feature data output from action data, according to one or more embodiments.

Referring to FIG. 3A, a feature set including a plurality of features may be extracted from each piece of action data included in a synthesis process. As an example, the features may be extracted based on keywords extracted from the action data. As an example, the features may be extracted from the action data by using a generative model. The method for extracting features from action data is not limited to the foregoing examples, and various methods may be used.

For example, a feature set including an action type, a substance type, the quantity of substance, a temperature, a duration, and a timestamp may be extracted from each piece of action data.

The action type and the substance type may belong to categorical features. The value of an action type feature may be determined as a predetermined label, such as ‘Add substance,’ ‘Set atmosphere,’ or ‘Set temperature,’ as non-limiting examples. The value of a substance type feature may be determined as an identification value (e.g., a name or a chemical formula) of substances, such as ‘A,’‘B,’‘vacuum,’or ‘nitrogen.’

The quantity of substance, temperature, duration, and timestamp may be numerical features. The values of numerical features may be determined to be numerical data, like natural numbers, integers, and/or real numbers.

In an embodiment, some types of features may not be extracted from a piece of action data. For example, a feature 311 of substance quantity type may not be extracted from third action data. The value of an unextracted feature may be determined to be a value (e.g., null) indicating that the feature is not extracted.

The feature set of FIG. 3A may be preprocessed to be converted into feature set data 302 shown in FIG. 3B.

Referring to FIG. 3B, a value of a feature 311 having a null value may be converted into a predetermined value (e.g., −1) through preprocessing. In an example, the numerical features (e.g., except for null values) may be normalized to have real numbers greater than or equal to 0 and less than or equal to 1. In an example, a value of a categorical feature may be changed to a mapped numerical value. For example, among the feature values of the action type, ‘Add substance’may be changed to 0, and ‘Set atmosphere’may be changed to 1.

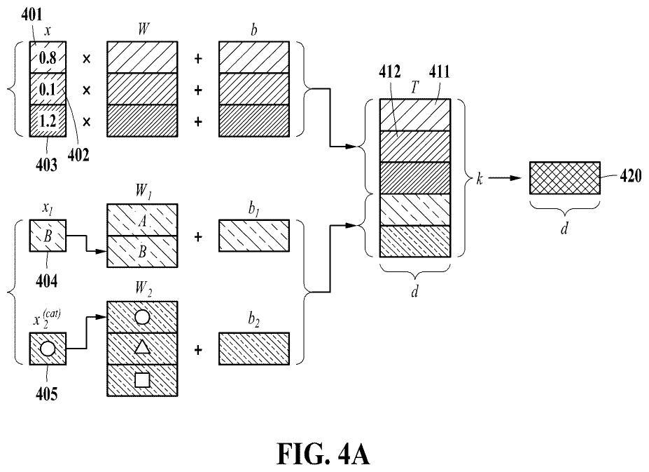

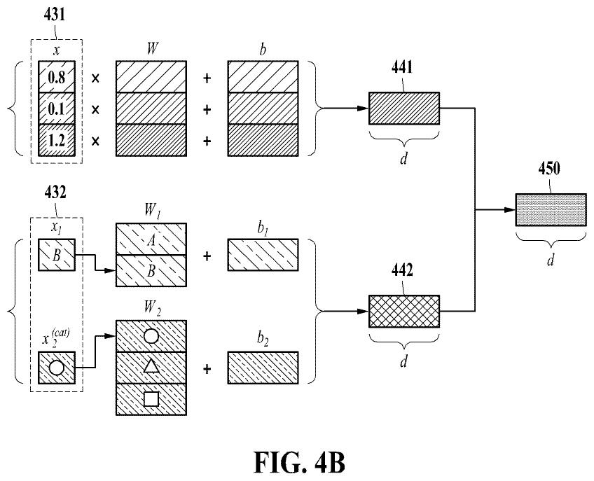

FIGS. 4A and 4B each illustrate an example of an operation of generating an embedding vector of a feature extracted from action data, according to one or more embodiments.

Referring to FIG. 4A, embedding data may be generated from each feature. For example, first embedding data 411 may be embedding data generated from a first feature 401, and second embedding data 412 may be embedding data generated from a second feature 402.

For example, in the case of numerical features, embedding data may be generated by multiplying a value x of each feature by a weight W and adding a bias b thereto. Weights and biases may be determined for each feature. As an example, a weight multiplied by the first feature 401 may be different from a weight multiplied by the second feature 402. As an example, a bias added to the first feature 401 may be different from a bias added to the second feature 402.

In an example, in the case of categorical features, a value of each feature may be changed to a mapped weight, and embedding data may be generated by adding a bias to the weight. Weights and biases may be determined for each feature. As an example, the weight mapped to a fourth feature 404 may be different from the weight mapped to a fifth feature 405. As an example, a bias b₁ added to the fourth feature 404 may be different from a bias b₂ added to the fifth feature 405.

The respective embedding data of different features may be generated to be in the same size. For example, the embedding data of the features 401 to 405 extracted from the action data may all be generated with a size of 1×d. The method of generating embedding data illustrated in FIG. 4A is only an example, and the method of generating embedding data may include various methods of generating a 1D feature into a d-dimensional vector.

The embedding data generated from each feature may be aggregated to be generated as a vector 420 of a certain size corresponding to the action data. For example, the aggregating of the embedding data generated from each feature may be performed by using various aggregation methods, such as sum, weighted sum, mean, weighted average, self-attention, and/or max pooling, of the embedding data generated from each feature.

FIG. 4B illustrates an example of hierarchical aggregation of features.

Referring to FIG. 4B, a first aggregate feature 441 may be generated by aggregating embedding data corresponding to some of the features extracted from action data. A second aggregate feature 442 may be generated by aggregating embedding data corresponding to some other features among the features extracted from the action data.

As an example, the first aggregate feature 441 may be generated by aggregating embedding data of features 431 belonging to numerical features. The second aggregate feature 442 may be generated by aggregating embedding data of features 432 belonging to the categorical feature.

A vector 450, which is the embedding data of the features extracted from the action data, may be generated by aggregating the first aggregate feature 441 and the second aggregate feature 442.

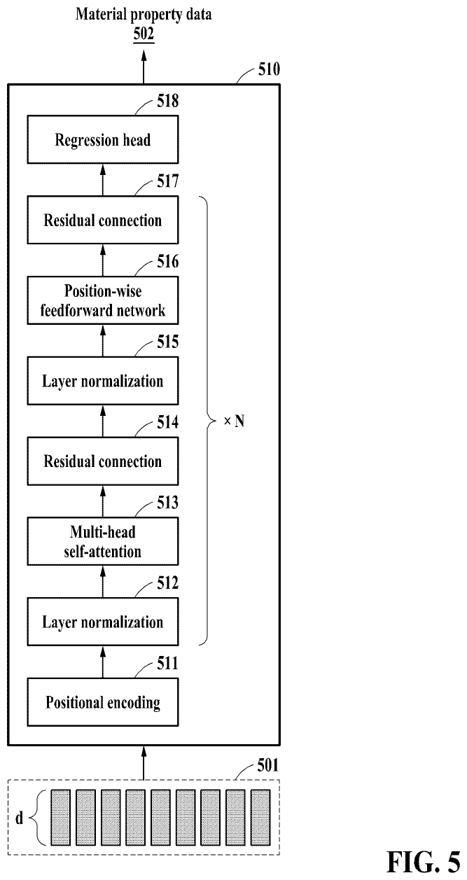

FIG. 5 illustrates an example of a transformer-based sequential data processing model, according to one or more embodiments.

Referring to FIG. 5, a sequential data processing model 510 may include a transformer-based model trained to predict a material property of an output of an input process from a vector sequence 501 corresponding to the input process.

The sequential data processing model 510 may be a model trained to receive the vector sequence 501 as an input and generate, as an output, material property data 502 based on the input.

The sequential data processing model 510 may perform a positional encoding operation 511 that encodes position information to reflect an order of input data. The sequential data processing model 510 may perform layer normalization operations 512 and 515 to normalize output values of each layer. The sequential data processing model 510 may perform a multi-head self-attention operation 513 to determine correlations between each token and other tokens of input data. The sequential data processing model 510 may perform residual connection operations 514 and 517 to add input values of each layer to an output of a next layer.

The sequential data processing model 510 may include a position-wise feedforward network 516, which is to apply, to an attention output, independent nonlinear transformation for each position.

The sequential data processing model 510 may include a regression head 518 for transforming a final output value to generate a value that the model is to predict. For example, the regression head 518 may be a layer that converts embedding data generated from the vector sequence 501 into the material property data 502, which is a final output value.

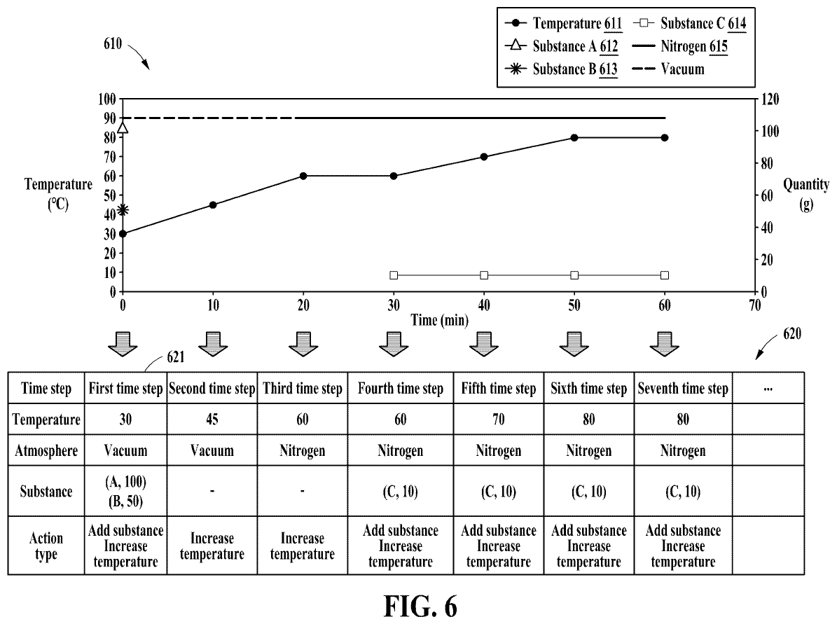

FIG. 6 illustrates an example of an operation of extracting features from action data for each time step, according to one or more embodiments.

Referring to FIG. 6, a synthesis process may include graph data 610. The graph data 610 may include a graph 611 indicating changes in temperature over time. The graph data 610 may include a graph 612 indicating a quantity of injection of a substance A at a specific time and a graph 613 indicating a quantity of injection of a substance B. The graph data 610 may include a graph 614 indicating changes in a quantity of a substance C over time. The graph data 610 may include a graph 615 indicating changes in an atmospheric condition over time. For example, an atmospheric condition in a first time step and a second time step may correspond to a vacuum state, and an atmospheric condition from a third time step onwards may correspond to a state that has changed to a nitrogen state.

Action data may correspond to data indicating a state of a specific time step of the graph data 610. Features 620 extracted from action data included in the synthesis process may include values of condition(s) corresponding to each time step (e.g., a 10-minute interval) included in the graph data 610. For example, features extracted from action data corresponding to a first time step 621, which is an interval from 0 to 10 minutes, may include a temperature of the first time step 621, atmosphere of the first time step 621, and a type and a quantity of substances (e.g., (A, 100), (B, 50)) added during the first time step 621.

In an example, the features extracted from action data may include action types determined based on changes in the graph. For example, a feature extracted from the action data corresponding to the first time step 621 may be determined as an action type of increasing temperature, since the temperature is increasing. For example, a feature extracted from the action data corresponding to the first time step 621 may be determined as adding a substance, since the quantity of the substance A is increasing.

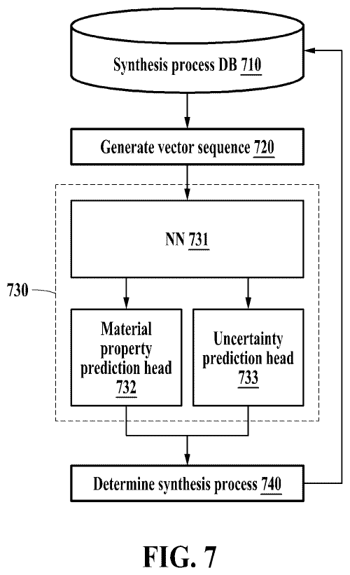

FIG. 7 illustrates an example of a method of determining a synthesis process to be input to a sequential data processing model from a synthesis process database, according to one or more embodiments. Operations of FIG. 7 may be performed in the sequence and manner as illustrated in FIG. 7. However, one or more of the operations may be performed in a different order, one or more of the operations may be omitted, two or more of the operations may be performed in parallel or simultaneously, and/or other operations may be additionally performed without departing from the spirit and scope of the described embodiments.

Referring to FIG. 7, a synthetic process database 710 may be a database that stores one or more different synthetic processes. The synthesis processes stored in the synthetic process database 710 may correspond to input data for performing the sequential data processing method described above. For example, the synthesis processes may correspond to input data of an electronic device that performs the sequential data processing method.

Operation 720 of generating a vector sequence from the synthesis processes stored in the synthesis process database 710 may be performed. Operation 720 of generating a vector sequence from a synthesis process may correspond to operations 110 and 120 of FIG. 1.

The generated vector sequence may be input to a sequential data processing model 730. The sequential data processing model 730 may include a neural network (NN) 731 that generates embedding data of a vector sequence, where the vector sequence is sequential data. For example, the neural network 731 may correspond to a portion of the sequential data processing model 510 of FIG. 5 excluding the regression head 518.

The sequential data processing model 730 may include a material property prediction head 732 and an uncertainty prediction head 733. The material property prediction head 732 may correspond to a first head, which is for material property prediction described above. The uncertainty prediction head 733 may correspond to a second head, which is for uncertainty prediction. The material property prediction head 732 may correspond to the regression head 518 in the sequential data processing model 510 of FIG. 5.

Based on an output of the material property prediction head 732 and an output of the uncertainty prediction head 733, operation 740 of determining a synthesis process to be input to the sequential data processing model 730 may be performed. Operation 740 of determining a synthesis process may include an operation of determining, among the synthesis processes stored in the synthesis process database 710, a next synthesis process to be input to the sequential data processing model 730. In an example, operation 740 of determining the synthesis process may include an operation of determining, among the synthesis processes stored in the synthesis process database 710, a next synthesis process from which a next vector sequence is to be generated in operation 720.

For example, the synthesis process to be input to the sequential data processing model 730 in the synthesis process database 710 may be determined based on an acquisition function predefined for the output of the material property prediction head 732 and the output of the uncertainty prediction head 733. For example, an acquisition function predefined for the output of the material property prediction head 732 and the output of the uncertainty prediction head 733 may be used to determine the next synthesis process to be input to the sequential data processing model 730 (and/or the next synthesis process from which a next vector sequence is to be generated in operation 720).

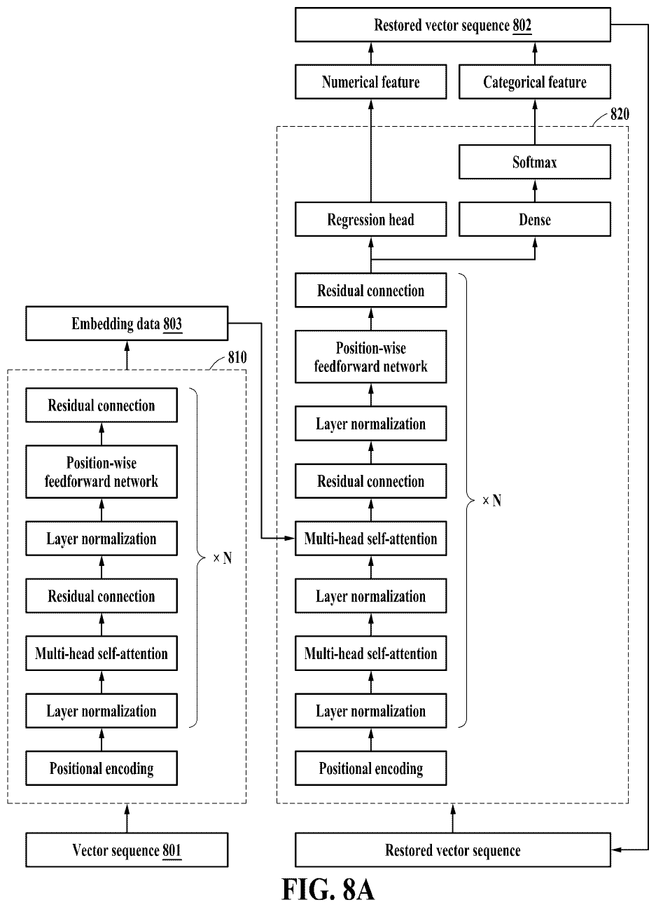

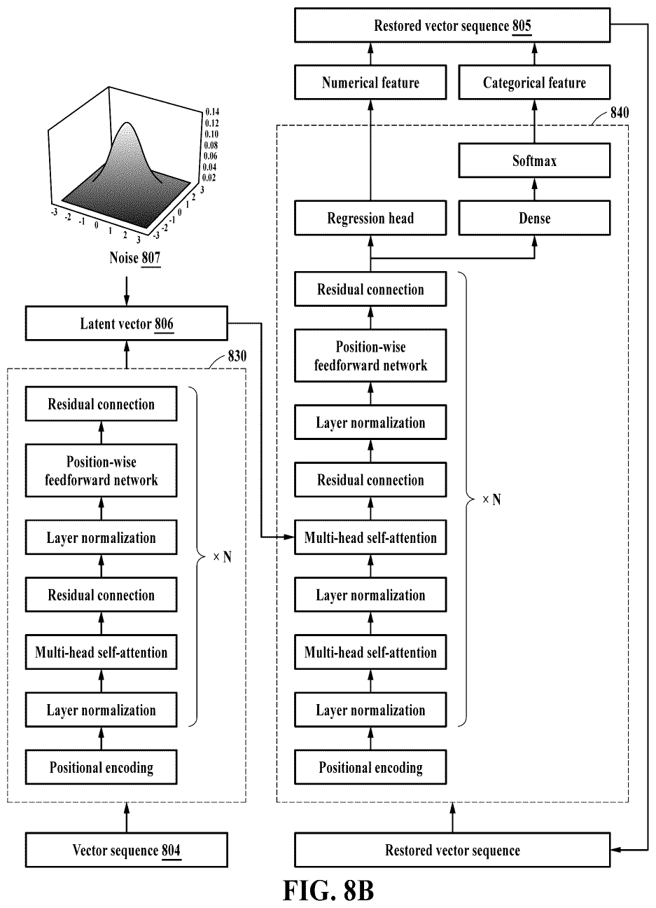

FIGS. 8A and 8B each illustrate an example of an autoencoder based on sequential data processing, according to one or more embodiments.

Referring to FIG. 8A, a vector sequence 801 generated by a sequential data processing method may be used for training the autoencoder. For example, the vector sequence 801 may include a vector sequence generated by operations 110 and 120 of FIG. 1.

The autoencoder may be a model trained to output a restored vector sequence 802 from the vector sequence 801 that has been input. The restored vector sequence 802 may be data generated by the autoencoder and may be the same data as the vector sequence 801 that has been input to the autoencoder.

An encoder 810 of the autoencoder may correspond to a model trained to generate embedding data 803 that is encoded data corresponding to the vector sequence 801. A decoder 820 of the autoencoder may be trained to restore the vector sequence 801 that has been input, based on the embedding data 803 that is encoded data output from the encoder 810 of the autoencoder.

The decoder 820 of the autoencoder may be trained to output the restored vector sequence 802 of the vector sequence 801 that has been input. The decoder 820 of the autoencoder may be trained to restore vectors in the input vector sequence 801 in time order. The decoder 820 of the autoencoder may predict an n-th vector of the input vector sequence 801, based on the vector sequence including up to the predicted n−1-th vector and the embedding data 803 output from the encoder 810. For example, the n-th vector may include one or more categorical features. For example, the n-th vector may include one or more numerical features. The vector sequence including up to the n-th vector and the embedding data 803 output from the encoder may be applied to the decoder 820 such that the n+1-th vector of the vector sequence 801 that has been input may be predicted.

For example, the autoencoder may be a transformer-based model. The encoder 810 of the autoencoder may correspond to a portion of the sequential data processing model 510 of FIG. 5 excluding the regression head 518. The decoder 820 of the autoencoder may be a transformer-based model.

Referring to FIG. 8B, a vector sequence 804 generated by the sequential data processing method may be used for training a variational autoencoder (VAE). For example, the vector sequence 804 may include the vector sequence generated by operations 110 and 120 of FIG. 1.

The VAE may be a model trained to learn a latent representation of the vector sequence 804 that has been input and to generate new data. For example, the trained VAE may generate a vector sequence corresponding to a new synthesis process from input noise. The input noise may include noise sampled from a multi-dimensional standard normal distribution.

An encoder 830 of the VAE may be trained to transform the vector sequence 804, which has been input, into a latent space. The encoder 830 of the VAE may sample a latent vector 806 in the latent space of the vector sequence 804 that has been input, based on noise 807 sampled from the multi-dimensional standard normal distribution.

The decoder 840 of the VAE may be trained to restore the vector sequence 804, which has been input, from the latent vector 806 sampled by the encoder 830. Similar to the training of the decoder 820 described above with reference to FIG. 8A, the decoder 840 of the VAE may be trained to output a restored vector sequence 805 of the input vector sequence 801.

The decoder 840 of the trained VAE may receive the noise 807 sampled from the multi-dimensional standard normal distribution as an input and may output a vector sequence. The vector sequence generated from noise in the decoder 840 may be a vector sequence corresponding to a new synthesis process, and vectors included in the vector sequence may include feature(s) corresponding to action data. For example, a vector sequence generated from the decoder 840 of the trained VAE may include “n” vectors corresponding to “n” pieces of action data. A k-th vector of the vector sequence generated from the decoder 840 of the trained VAE may include one or more categorical features corresponding to the k-th action data of the synthesis process. The k-th vector generated from the decoder 840 of the trained VAE may include one or more numerical features corresponding to the n-th action data of the synthesis process.

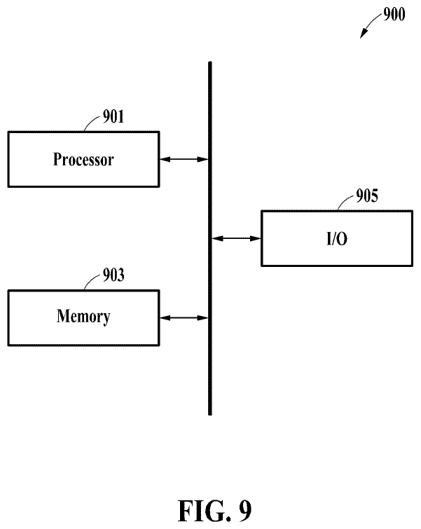

FIG. 9 illustrates an example of a configuration of an electronic device, according to one or more embodiments.

Referring to FIG. 9, an electronic device 900 may include a processor 901 (e.g., one or more processors), a memory 903 (e.g., one or more memories), and an input/output (I/O) device 905. The electronic device 900 may include an electronic device that performs the sequential data processing method described above with reference to FIGS. 1 to 8B. For example, the electronic device 900 may include at least one of a server and a terminal (e.g., a personal computer (PC), a smartphone, a tablet, and/or a wearable device).

The processor 901 may perform at least one operation of the sequential data processing method described above with reference to FIGS. 1 to 8B.

For example, the processor 901 may perform at least one of extracting features from action data in a time order included in a synthesis process, generating a vector sequence corresponding to the action data based on embedding data of features extracted from the action data, and generating data related to a synthesis process generated from encoded data of the vector sequence by applying the vector sequence to a sequential data processing model.

For example, the processor 901 may perform at least one of extracting features from action data in a time order included in a synthesis process, generating a vector sequence corresponding to the action data based on embedding data of features extracted from the action data, and training an autoencoder based on sequential data processing to output a vector sequence from the vector sequence based on encoded data of the vector sequence.

The memory 903 may be a volatile memory or a non-volatile memory and may store data related to the sequential data processing method described above with reference to FIGS. 1 to 8B. For example, the memory 903 may store data generated during an execution of the sequential data processing method or data required to perform the sequential data processing method. For example, the memory 903 may store a sequential data processing model. For example, the memory 903 may store at least one parameter determined during a training process of the sequential data processing model. For example, the memory 903 may include a synthesis process database.

The memory 903 may not be a component of the electronic device 900 but may be included in an external device accessible by the electronic device 900. In this case, the electronic device 900 may receive data stored in the memory 903 included in the external device and transmit data to be stored in the memory 903 via a communication device.

The memory 903 may store a program implementing the sequential data processing method described above with reference to FIGS. 1 to 8B. The processor 901 may execute a program stored in the memory 903 and control the electronic device 900. Code from the program executed by the processor 901 may be stored in the memory 903. For example, the memory 903 may be or include a non-transitory computer-readable storage medium storing instructions that, when executed by the processor 901, configure the processor 901 to perform any one, any combination, or all of the operations and/or methods described herein with reference to FIGS. 1-9.

The memory 903 may store instructions, which, when executed by the processor 901, may cause the electronic device 900 to extract features from action data, which is in a time order, included in a synthesis process, generate a vector sequence corresponding to the action data based on embedding data of the features extracted from the action data, and generate data regarding the synthesis process generated from encoded data of the vector sequence by applying the vector sequence to a sequential data processing model.

The electronic device 900 may include the I/O device 905 including an input device and an output device. The electronic device 900 may be connected to an external device (e.g., a PC or a network) and exchange data with the external device through the I/O device 905. For example, the electronic device 900 may receive synthesis process data and output physical data, which is an output of sequential data processing, through the I/O device 905.

The electronic device 900 may further include other components not shown in the drawings. For example, the electronic device 900 may include a communication device. The communication device may provide a function for the electronic device 900 to communicate with other electronic devices or other servers through a network. In addition, the electronic device 900 may further include other components such as, for example, a transceiver, various sensors, and a database.

The electronic devices, processors, memories, I/O devices, electronic device 900, processor 901, memory 903, and I/O device 905 described herein, including descriptions with respect to respect to FIGS. 1-9, are implemented by or representative of hardware components. As described above, or in addition to the descriptions above, examples of hardware components that may be used to perform the operations described in this application where appropriate include controllers, sensors, generators, drivers, memories, comparators, arithmetic logic units, adders, subtractors, multipliers, dividers, integrators, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit (ALU), a digital signal processor (DSP), a microcomputer, a programmable logic controller, a field-programmable gate array (FPGA), a programmable logic array (PLU), a microprocessor, or any other device or combination of devices that is configured to respond to and execute instructions (e.g., code or coding) in a defined manner to achieve a desired result. In one example, a processor or computer includes, or is connected to, one or more memories storing the instructions or software that are executed by the processor or computer. Hardware components implemented by a processor or computer may execute the instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both, and thus while some references may be made to a singular processor or computer, such references also are intended to refer to multiple processors or computers. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. As described above, or in addition to the descriptions above, example hardware components may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing. Thus, references to a processor herein mean processing circuitry (e.g., circuitry that includes one or more processing element(s) circuits). One or more processors comprising processing circuitry also refers to each processor comprising processing circuitry, as well as some or all of the one or more processors comprising the same processing circuitry. In addition, processors(s) and controller(s), as a non-limiting example, do not mean human processing or human control, but rather, refer to hardware components as described herein, as non-limiting examples.

The methods illustrated in, and discussed with respect to, FIGS. 1-9 that perform the operations described in this application are performed by computing hardware, for example, by one or more processors or computers, implemented as described above implementing the instructions (e.g., computer or processor/processing device readable instructions) or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations. References to a processor, or one or more processors, as a non-limiting example, configured to perform two or more operations refers to a processor or two or more processors being configured to collectively perform all of the two or more operations, as well as a configuration with the two or more processors respectively performing any corresponding one of the two or more operations (e.g., with a respective one or more processors being configured to perform each of the two or more operations, or any respective combination of one or more processors being configured to perform any respective combination of the two or more operations). Likewise, a reference to a processor-implemented method is a reference to a method that is performed by one or more processors or other processing or computing hardware of a device or system.

The instructions or software to control computing hardware, for example, one or more processors or computers, to implement the hardware components and perform the methods as described above may be written as computer programs, code segments, or other executable instructions or any combination thereof, for individually or collectively instructing or configuring the one or more processors or computers to operate as a machine or special-purpose computer to perform the operations that are performed by the hardware components and the methods as described above. In one example, the instructions or software include machine code that is directly executed by the one or more processors or computers, such as machine code produced by a compiler. In another example, the instructions or software includes higher-level code that is executed by the one or more processors or computer using an interpreter. The instructions or software may be written using any programming language based on the block diagrams and the flow charts illustrated in the drawings and the corresponding descriptions herein, which disclose algorithms for performing the operations that are performed by the hardware components and the methods as described above.

The instructions or software to control computing hardware, for example, one or more processors or computers, to implement the hardware components and perform the methods as described above, and any associated data, data files, and data structures, may be recorded, stored, or fixed in or on one or more non-transitory computer-readable storage media, and thus, not a signal per se. Thus, references herein to storage media mean storage media hardware, and does not mean transitory media, nor a signal per se. As described above, or in addition to the descriptions above, examples of a non-transitory computer-readable storage medium include one or more of any of read-only memory (ROM), random-access programmable read only memory (PROM), electrically erasable programmable read-only memory (EEPROM), random-access memory (RAM), dynamic random access memory (DRAM), static random access memory (SRAM), flash memory, non-volatile memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, blue-ray or optical disk storage, hard disk drive (HDD), solid state drive (SSD), flash memory, a card type memory such as a multimedia card or a micro card (for example, secure digital (SD) or extreme digital (XD)), magnetic tapes, floppy disks, magneto-optical data storage devices, optical data storage devices, hard disks, solid-state disks, and/or any other device that is configured to store the instructions or software and any associated data, data files, and data structures in a non-transitory manner and provide the instructions or software and any associated data, data files, and data structures to one or more processors or computers so that the one or more processors or computers can execute the instructions. In one example, the instructions or software and any associated data, data files, and data structures are distributed over network-coupled computer systems so that the instructions and software and any associated data, data files, and data structures are stored, accessed, and executed in a distributed fashion by the one or more processors or computers.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

Therefore, in addition to the above and all drawing disclosures, the scope of the disclosure is also inclusive of the claims and their equivalents, i.e., all variations within the scope of the claims and their equivalents are to be construed as being included in the disclosure.

What is claimed is:

1. A processor-implemented method comprising:
extracting features from action data included in a synthesis process, the action data being in a time order;
generating a vector sequence corresponding to the action data based on embedding data of the features extracted from the action data; and
generating data regarding the synthesis process generated from encoded data of the vector sequence, by applying the vector sequence to a sequential data processing model.

2. The method of claim 1, wherein the generating of the data regarding the synthesis process comprises generating material property data that is predicted as a result of the synthesis process, by applying the vector sequence to the sequential data processing model.

3. The method of claim 1, wherein the generating of the vector sequence comprises generating the embedding data of the features extracted from the action data, based on preprocessing of the features extracted from the action data.

4. The method of claim 3, wherein the preprocessing of the features comprises any one or any combination or any two or more of:
determining a value of an unextracted feature;
normalizing numerical features; and
encoding categorical features.

5. The method of claim 1, wherein the sequential data processing model comprises a transformer-based model trained to predict a material property of an output of an input process from a vector sequence corresponding to the input process.

6. The method of claim 1, wherein the extracting of the features from the action data comprises extracting the features from the action data for each time step included in the synthesis process, the synthesis process being time-series data.

7. The method of claim 1, wherein the extracting of the features comprises extracting predetermined type of feature from each piece of the action data.

8. The method of claim 1, wherein the generating of the vector sequence comprises:
generating, as the embedding data, embedding data of each feature extracted from the action data;
generating a vector corresponding to each piece of the action data by aggregating the embedding data of each feature; and
generating the vector sequence comprising the vector corresponding to each piece of the action data.

9. The method of claim 8, wherein the generating of the vector corresponding to each piece of the action data comprises:
generating a first aggregate feature by aggregating embedding data corresponding to one or more features extracted from each piece of the action data;
generating a second aggregate feature by aggregating embedding data corresponding to one or more features extracted from each piece of the action data; and
aggregating the first aggregate feature and the second aggregate feature.

10. The method of claim 1, wherein the sequential data processing model comprises:
a first head for material property prediction; and
a second head for uncertainty prediction, and the generating of the data regarding the synthesis process comprises:
generating an output of the first head and an output of the second head corresponding to the encoded data of the vector sequence; and
determining, from a synthesis process database, another synthesis process to be input to the sequential data processing model, based on an acquisition function predefined for the output of the first head and the output of the second head.

11. The method of claim 1, further comprising training an autoencoder based on sequential data processing to output a restored vector sequence of the vector sequence based on the encoded data of the vector sequence.

12. A processor-implemented method comprising:
extracting features from action data in a time order included in a synthesis process;
generating a vector sequence corresponding to the action data based on embedding data of the features extracted from the action data; and
training an autoencoder based on sequential data processing to output a restored vector sequence of the vector sequence based on encoded data of the vector sequence.

13. The method of claim 11, wherein the training of the autoencoder comprises:
training an encoder of the autoencoder to generate the encoded data of the vector sequence, and
for the outputting of the restored vector sequence, training a decoder of the autoencoder to restore the vector sequence from the encoded data of the vector sequence generated by the encoder.

14. The method of claim 11, wherein the training of the autoencoder comprises:
applying, to a decoder of the autoencoder, a latent vector sampled from a latent space of the vector sequence output from an encoder of the autoencoder, based on noise obtained according to a predetermined probability distribution; and
for the outputting of the restored vector sequence, training the decoder of the autoencoder to restore the vector sequence from the latent vector.

15. The method of claim 13, wherein the decoder of the trained autoencoder is configured to generate a vector sequence corresponding to an arbitrary synthesis process from arbitrary noise obtained according to a probability distribution.

16. An electronic device comprising:
one or more processors comprising processing circuitry; and
memory comprising one or more storage media storing instructions that, when executed individually or collectively by the one or more processors, cause the electronic device to:
extract features from action data included in a synthesis process, the action data being in a time order;
generate a vector sequence corresponding to the action data based on embedding data of the features extracted from the action data; and
generate data regarding the synthesis process generated from encoded data of the vector sequence, by applying the vector sequence to a sequential data processing model.

17. The electronic device of claim 15, wherein, for the generating of the data regarding the synthesis process, the execution of the instructions causes the electronic device to generate material property data that is predicted as a result of the synthesis process, by applying the vector sequence to the sequential data processing model.

18. The electronic device of claim 15, wherein, for the generating of the vector sequence, the execution of the instructions causes the electronic device to generate the embedding data of the features extracted from the action data, based on preprocessing of the features extracted from the action data.

19. The electronic device of claim 15, wherein, for the extracting of the features from the action data, the execution of the instructions causes the electronic device to extract the features from the action data for each time step included in the synthesis process, the synthesis process being time-series data.

20. The electronic device of claim 15, wherein
the sequential data processing model comprises:
a first head for material property prediction; and
a second head for uncertainty prediction, and
for the generating of the data regarding the synthesis process, the execution of the instructions causes the electronic device to:
generate an output of the first head and an output of the second head corresponding to the encoded data of the vector sequence; and
determine, from a synthesis process database, another synthesis process to be input to the sequential data processing model, based on an acquisition function predefined for the output of the first head and the output of the second head.
